(19) United States
(12) Patent Application Publication
(10) Pub. No.: US 2015/0158877 A1
(43) Pub. Date: Jun. 11, 2015

(54) DUALISTIC SUBSTITUTED PHTHALOCYANINE ZINC FORMULA, PROCESS OF SEPARATING THE SAME AND USE THEREOF

(71) Applicants: Naisheng CHEN, Fuzhou (CN); Jining Huang, Fuzhou (CN); Jinping Xue, Fuzhou (CN); Hong Liu, Fuzhou (CN); Jian Wang, Fuzhou (CN); Jundong Wang, Fuzhou (CN); Zhou Jiang, Fuzhou (CN)

(72) Inventors: Naisheng CHEN, Fuzhou (CN); Jining Huang, Fuzhou (CN); Jinping Xue, Fuzhou (CN); Hong Liu, Fuzhou (CN); Jian Wang, Fuzhou (CN); Jundong Wang, Fuzhou (CN); Zhou Jiang, Fuzhou (CN)

(73) Assignee: FUZHOU UNIVERSITY, Fuzhou (CN)

(21) Appl. No.: 14/469,809

(22) Filed: Aug. 27, 2014

Related U.S. Application Data
(63) Continuation-in-part of application No. 13/025,380, filed on Feb. 11, 2011, now abandoned.

(51) Int. Cl. C07D 487/22

(52) U.S. Cl. CPC C07D 487/22

(57) ABSTRACT

The invention discloses a pharmaceutical formula used in photodynamic therapy (PDT) for cancer or precancerous lesions. The formula includes four components of disulfonic acid diphthaloyl iminomethyl dualistic substituted phthalocyanine zinc alkali salt. Furthermore, this invention also provides an industrial chromatography separation process. These components is obtained by separating ten cis isomers of ZnPcS2P2 from synthetic products and then separating among them 4 isomers with significant amphiphilic property. It is evident that 4 isomers of the invention shows improved drug affect compared to formulas containing those 10 cis-isomers.

### CROSS-REFERENCE TO RELATED APPLICATIONS

This Continuation-In-Part Application claims the benefit of co-pending U.S. Non-Provisional Patent Application Ser. No. 13/025,380, filed on Feb. 11, 2011, which is incorporated herein in their entirety.

### BACKGROUND OF THE INVENTION

1. Field of the Invention

The present invention relates to a dualistic substituted phthalocyanine zinc formula, a process of separating the same and a use thereof. More particularly, the present invention relates to a phthalocyanine zinc formula, which can be used as a photosensitizer in photodynamic therapy (PDT).

2. Description of the Prior Art

Intensive researches on phthalocyanine compounds as photosensitizers in photodynamic therapy (PDT) for diseases such as cancers have been widely made in recent years. For example, CN Patent application, application number: 200410013492.4, discloses a method of preparing phthalocyanine metal complexes which uses 4-sulfonic acid potassium phthalonitrile and 4-phthaloyl iminomethyl phthalonitrile as synthetic products. First of all, within a lithium solution in alcohol, sulfonic acid potassium substituted semi-phthalocyanine and phthaloyl iminomethyl substituted semi-phthalocyanine are formed as precursors. Then, cyclization reaction is carried out in 2-ethoxyethanol to form bis(sulfonic acid potassium)bis(phthaloyl iminomethyl) lithium phthalocyanine zinc intermediates which then react with zinc salts in DMF to form amphiphilic phthalocyanine zinc complexes.

The obtained phthalocyanine zinc formula from the above synthesis contains isomers of complicate structures and components. For example, disulfonic acid potassium phthaloyl iminomethyl phthalocyanine zinc contains 10 cis-isomers and 5 trans-isomers. In this disclosed content of the present invention, disulfonic acid potassium phthaloyl iminomethyl phthalocyanine zinc and the salt thereof are referred to as ZnPcS₂P₂. There may be some varieties in definition of the term ZnPcS₂P₂ with different substituent groups in similar forms. For example, if a ring of phthalocyanine zinc has 3 —S substituent groups and 1 —P substituent group, it is referred to as ZnPcS₃P. If the ring of phthalocyanine zinc has 2 —S substituent groups and 2 —P substituent groups, it is referred as to ZnPcS₂P₂. Therefore, formula of phthalocyanine zinc can be ZnPcS₄, ZnPcP₄, ZnPcSP₃ etc., depending on the number of —S and —P substituent groups. The —S substituent group can be sulfonic acid group or salts thereof. The —P substituent group can be phthaloyl iminomethyl. However, the difference in photodynamic therapy effect between various isomers of phthalocyanine zinc has not been studied yet. No process of separating specific isomers with significant effect in photodynamic therapy from the isomer mixture has been proposed, neither.

The present invention is therefore intended to obviate or at least alleviate the problems encountered in prior art, especially in terms of flexibility of special arrangement.

### SUMMARY OF THE INVENTION

It is an objective of the present invention to provide a dualistic substituted phthalocyanine zinc formula used in photodynamic therapy for cancer or precancerous lesions, an use of phthalocyanine zinc in pharmaceutical application, and an application of phthalocyanine zinc to an industrial chromatography separation process.

It discloses a pharmaceutical formula used in photodynamic therapy (PDT) for cancer or precancerous lesions. The formula includes four components of disulfonic acid diphthaloyl iminomethyl dualistic substituted phthalocyanine zinc alkali salt. Furthermore, this invention also provides an industrial chromatography separation process. These components is obtained by separating ten cis isomers of ZnPcS2P2 from synthetic products and then separating among them 4 isomers with significant amphiphilic property. It is evident that 4 isomers of the invention shows improved drug affect compared to formulas containing those 10 cis-isomers.

In order to achieve the above and other objectives, the invention provides a dualistic substituted phthalocyanine zinc formula, the above dualistic substituted phthalocyanine zinc formula includes the following four isomers of phthalocyanine zinc:

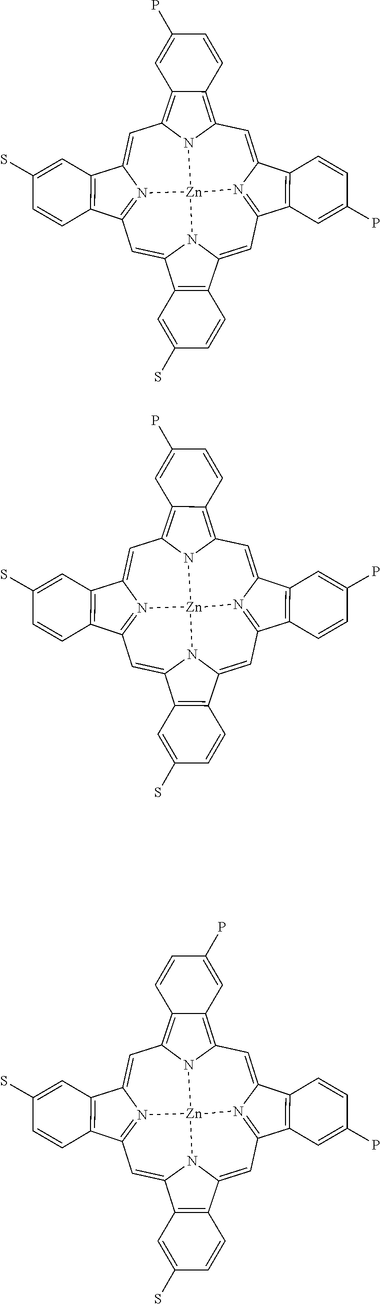

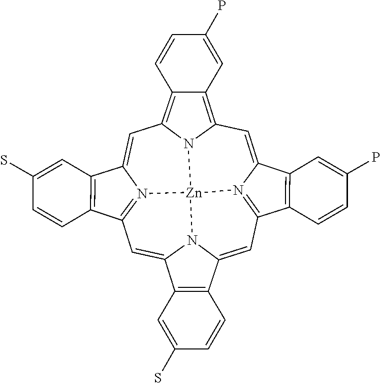

the above dualistic substituted phthalocyanine zinc formula are isomers of ZnPcS₂P₂, wherein Zn is divalent zinc ions, Pc is phthalocyanine ring base, —S is —SO₃⁻M⁺, and SO₃⁻ is sulfo-group, M⁺ is a pharmaceutically acceptable alkali metal cation,

—P is

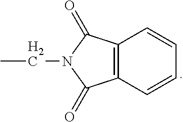

The invention further provides an use of a dualistic substituted phthalocyanine zinc formula used in photodynamic therapy for cancer or precancerous lesions, the above dualistic substituted phthalocyanine zinc formula comprises the following four isomers of phthalocyanine zinc:

the above dualistic substituted phthalocyanine zinc formula are isomers of ZnPcS₂P₂, wherein Zn is divalent zinc ions, Pc is phthalocyanine ring base; —S is —SO₃⁻M⁺, and SO₃⁻ is sulfo-group, M⁺ is a pharmaceutically acceptable alkali metal cation,

—P is

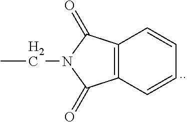

The invention also provides a separation process of dualistic substituted phthalocyanine zinc formula, characterized in that the process comprises the following steps:

Step 1: separating ten cis isomers of ZnPcS₂P₂ from synthetic products comprised of ZnPcS₄, ZnPcS₃P, ZnPcS₂P₃, and ZnPcP₄, wherein Zn is divalent zinc ions, Pc is phthalocyanine ring base; S is —SO₃⁻M⁺, and SO₃⁻ is sulfo-group, M⁺ is a pharmaceutically acceptable alkali metal cation;

P is

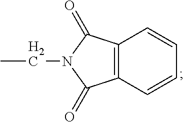

Step 2: separating the above four isomers of dualistic substituted phthalocyanine zinc of claim 1 from the above ten cis isomers of ZnPcS₂P₂.

In the process of dualistic substituted phthalocyanine zinc formula, the separation process use industrial chromatographic column separation method, wherein chromatographic column fillers are revers-phase C₁₈ silica gels, the above fillers pore diameter is 100 A to 120 A, the above fillers carbon load is 17% to 19%, the above fillers diameter is 10-50 umm, column temperature is 20° C. to 35° C., the eluant is a 65%˜70% (volume) DMF aqueous solution with buffer, the above eluant pH is 7.0 to 8.0.

The effect of the invention is superior to the existing technology. The use of mixture of four dualistic substituted phthalocyanine zinc monomers according to the present invention offers significantly improved photodynamic effect in terms of cancer cell growth inhibition, compared to an original mixture of 15 isomers including cis- and trans-monomers, or a mixture of 10 cis-monomers. Please see the following experiments and comparative examples.

Photodynamic Activity of Tumor Cells in vitro with Comparative Experiments

By using MTT (tetrazolium salt) reduction on tumor cells which have been treated by a photosensitizer and radiated by 670 nm laser, the effect of the concentration of the photosensitizer on inhibition of tumor cell growth is illustrated by dotting a dose-response curves and calculating the median effective concentration (IC₅₀). The results are shown in Table 1.

The inhibition effect of ZnPc series photosensitizers on the tumor cells in vitro is compared. In the table 1, the 4 cis-isomers are the formula of the 4 dualistic substituted phthalocyanine zinc isomers as provided by the invention. The 10 isomers are meant to be 10 possible cis-isomers of ZnPcS₂P₂. The trans-isomers are meant to be all possible trans-isomers of ZnPcS₂P₂.

TABLE 1

| | i. IC50 (μM) | | |
|---|---|---|---|
| Cell | Cis-(10 | Cis-(4 | |
| strains | isomers) | isomer) | Trans- |
| HELF | b. 0.46 | e. 0.044 | 0.58 |
| (Human | c. 0.042 | f. 0.039 | 0.093 |
| embryonic | d. 0.010 | g. 0.0027 | 0.017 |
| lung | | | |
| fibroblasts) | | | |
| BGC823 | | | |
| (Human | | | |
| gastric | | | |
| cancer | | | |
| cells ) | | | |
| K562 | | | |
| (Human | | | |
| leukemia | | | |
| cells) | | | |

From Table 1, among the formulas of photosensitizers (10 cis-isomer formula, 4cis-isomer formula and trans-isomer formula) in terms of required concentration for 50% inhibition (IC₅₀) on growth of three cancer cells, the 4 cis-isomer formula shows the best inhibition effect.

Other objectives, advantages and features of the present invention will become apparent from the following description referring to the attached drawings.

### BRIEF DESCRIPTION OF THE DRAWINGS

The present invention will be described via detailed illustration of the preferred embodiment versus prior art referring to the drawings where:

FIG. 1 is a HPLC graph of dualistic substituted phthalocyanine zinc mixture obtained by “semi-phthalocyanine ring synthetic method” according to a first embodiment of the invention.

FIG. 2 is a HPLC graph of roughly separated fractions collected in a first embodiment of the invention.

FIG. 3 is a HPLC graph of finely separated fractions collected in a first embodiment of the invention.

FIG. 4 is a HPLC graph of finely separated fractions collected in a second embodiment of the invention.

FIG. 5 is a HPLC graph of finely separated fractions collected in a third embodiment of the invention.

### DETAILED DESCRIPTION OF THE INVENTION

All terms in the content of this specification are described below.

1. All the liquids—concentrations of liquid components of liquid immiscible system are indicated in volume. For example, 65% DMF aqueous solution means the volume of DMF in the solution is 65%;
2. In description of the synthetic products and the fillers, the weight of the fillers means the dry weight thereof;
3. In the course of the whole separation process, the temperature is controlled in the range of normal room temperature, that is 20˜35° C. If the temperature falls out of this range, viscosity and other parameters of the solution will change, causing failure to reach effects as described in the embodiment;
4. HPLC analysis of intermediates and final products:

1) Chromatographic conditions for analysis of synthesized product components:

Chromatographic conditions for column Luna 15 cm*0.46 mm

5 μmm: column temperature of 30° C.,

| | | | % A | |
|---|---|---|---|---|
| | Time | Flow | 10 mM TEA | % B |
| | (min) | (ml/min) | pH≈5.1) | (CH₃CN:DMF = 30%:70%) |
| 1 | 0.01 | 1.00 | 95 | 5 |
| 2 | 5.00 | 1.00 | 95 | 5 |
| 3 | 25.00 | 1.00 | 0 | 100 |
| 4 | 30.00 | 1.00 | 0 | 100 |

In the spectra, ZnPcS₄ can be recognized from the peak of 15˜16 min, ZnPcS₃P can be recognized from the peaks of 19˜21 min, trans ZnPcS₂P₂ can be recognized from the peak of 23 min, cis-ZnPcS₂P₂ can be recognized from the peak of 23.5˜25 min, ZnPcSP₃ can be recognized from the peak of 27 min peak, and ZnPcP₄ can be recognized from the peak of 30.5 min.

2) Chromatographic conditions for analysis of the roughly separated product components

Chromatographic column Shiseido CAPCELL PAK C₁₈ 15 cm*0.46 cm 5 μmm, chromatographic conditions are as follows: column temperature 30° C.,

| | | | % A | % B |
|---|---|---|---|---|
| | Time | Flow | (20 mM | (THF:MeOH:DMF = |
| | (min) | (ml/min) | TEA pH≈5.1) | 15%:10%:75%) |
| 1 | 0.01 | 1.00 | 53 | 47 |
| 2 | 120.00 | 1.00 | 53 | 47 |

In the spectra, 4 peaks between 40˜65 min indicate 4 target products of cis ZnPcS₂P₂.

### Example 1

### Synthetic Products:

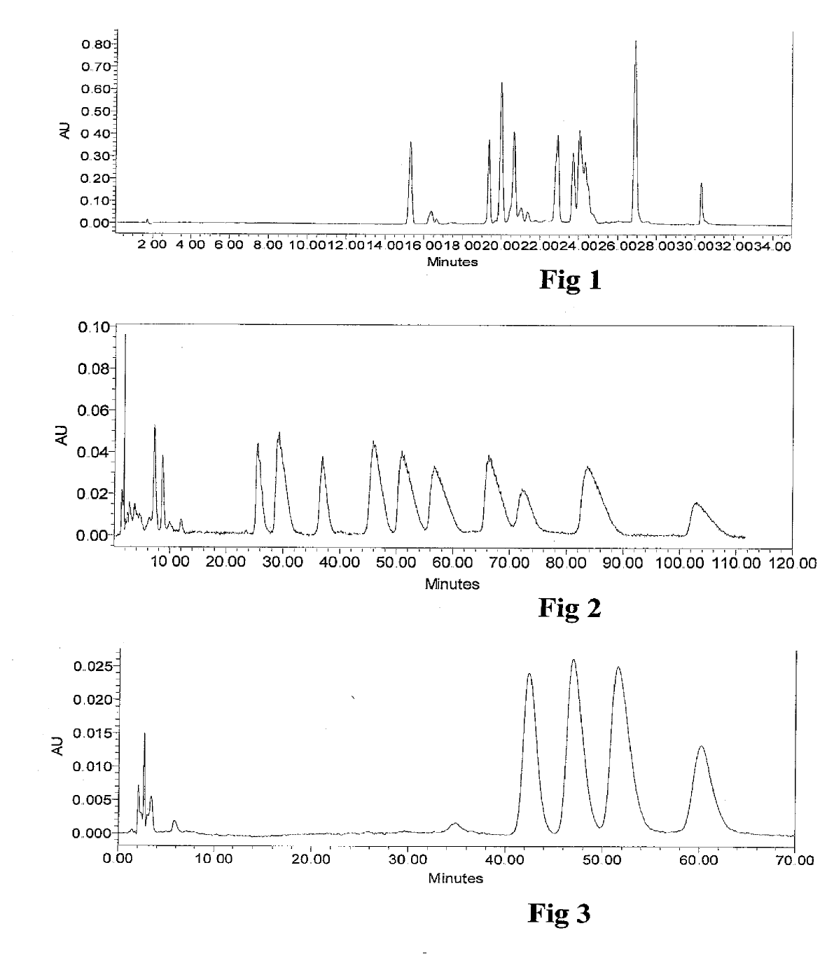

A “dualistic substituted semi-phthalocyanine ring synthetic method” is used to prepare dualistic substituted phthalocyanine zinc mixture. Also see CN patent application No. “200410013492.4”. The dualistic substituted phthalocyanine zinc mixture contains cis- and trans-isomers of ZnPcS₂P₂, 15 in total, and ZnPcS₄, ZnPcS₃P, ZnPcSP₃ and ZnPcP₄, along with components of other synthetic products and intermediates thereof. HPLC graph of the obtained products are shown in FIG. 1.

1) Rough separation:
a. Balancing through a rough separation column: the rough separation column has a packed length of 300 mm and a diameter of 50 mm, and fillers are Japanese YMC*Exphere C₁₈ 50 μm,
b. Injection: take a certain amount of the synthetic products to make 65% DMF aqueous solution of pH 7.0 to 8.0. The 65% DMF aqueous solution is pumped into the rough separation column. In the above solution, the content of contains dualistic substituted phthalocyanine zinc content is 0.5% of filler weight. The concentration of dualistic substituted phthalocyanine zinc is 8 g/L. Injection column temperature is 20° C. The injection flow rate=100 ml/min.
c. Elution: pH 7.0˜8.0 DMF aqueous solutions at different concentrations are used in turns for elution. The concentration of DMF aqueous solutions are in the range of 10% to 100%. During the elution, a low-concentration DMF aqueous solution is first pumped into the column and then a high-concentration of DMF solution.

Specifically, a mixture of 10% DMF-90% water is first used for 10-min elution. Thereafter a mixture of 30% DMF-70% water is used to elution for 20 minutes. Then a mixture of 50% DMF-50% water is used for 5 minutes. A mixture of 63% DMF-37% water is used in turns for 15 minutes. Then the mixture is changed to be 65% DMF-35% water and used for further 5 minutes. Finally, 100% DMF is used to wash the column for 10 minutes. The elution flow rate maintains the same, namely the flow rate 100 ml per minute, during the whole process.

The components of the eluent are correspondingly collected and, cis-enriched fractions of dualistic substituted phthalocyanine zinc are obtained. The eluting column temperature is 20° C. The HPLC results of the collected fractions are shown in FIG. 2.

a) Column enrichment: The cis-enriched fractions of dualistic substituted phthalocyanine zinc obtained at the step 1c) are pumped into an enrichment column which has fillers the same as those used in the rough separation. Then a 10%˜30% (volume) DMF aqueous solution and a 70%˜80% (volume) DMF aqueous solution, both being the same pH value as those used in injection step. The enriched fractions of dualistic substituted phthalocyanine zinc are collected.

2) Fine separation:
a) Balancing through a fine separation column: the column has a packed length of 700 mm and a diameter of 100 mm. The fillers are Japanese YMC*Gel Exphere C₁₈ 10 μm (alternatively YMC*Gel Exphere C₁₈ 20 μm);
b) Injection: take the roughly separated substances to make a 60% DMF aqueous solution with pH 8.0. The aqueous solution is pumped into the fine separation column. In the above solution, the content of dualistic substituted phthalocyanine zinc is 0.10% of filler weight. The concentration of dualistic substituted phthalocyanine zinc is 5 g/L. The injection column temperature is 20° C. The injection flow rate is 80 ml/min;
c) Elution: pH 8.0˜8.2, 60% DMF aqueous solution is used as a flowing phase for elution in the fine separation column. The eluting column temperature is 20° C. The elution flow rate is 200 ml/min. According to the formula of the eluent used here, four isomer-enriched fractions of dualistic substituted phthalocyanine zinc are collected.
d) Solvent Replacement: The fractions obtained at the step 2 c) are pumped into a replacement column. The fillers are the same as those used in for fine separation column. A 65% acetonitrile aqueous solution, having the same pH value as that used for injection, is used for elution. Target fractions are then collected.

3) Column regeneration after separation (rough separation column and fine separation column), by using the following solutions in turns:

ii. The column is washed by using a 90% DMF aqueous solution until it is colorless. A 10% DMF aqueous solution is used for 2 to 3 column washing cycles. A 10% DMF solution (adjusted with acetic acid until the pH value becomes 2-3) is used for 2 to 3 column washing cycles. A 10% methanol aqueous solution is then used. Finally, 100% methanol is used.

### Example 2

The steps are the same as those in Example 1, except that some conditions are changed as follows.

The synthetic products are unchanged.

1) Rough separation:
a) Balancing through a rough separation column: the rough separation column has a packed length of 600 mm and a diameter of 100 mm, and fillers are Japanese Daisogel C₁₈ 50 μm,
b) Injection: take a certain amount of the synthetic products to make 70% DMF aqueous solution of pH 7.0 to 8.0. The 70% DMF aqueous solution is pumped into the rough separation column. In the above solution, the content of contains dualistic substituted phthalocyanine zinc content is 0.20% of the weight of the fillers. The concentration of dualistic substituted phthalocyanine zinc is 12 g/L. Injection column temperature is 28° C. The injection flow rate=400 ml/min.
c) Elution: The eluting column temperature is 28° C. The remaining conditions are the same as those in Example 1.
d) Column enrichment: A 20% DMF aqueous solution and a 75% (volume) DMF aqueous solution are used in turns for elution. The enriched fractions of dualistic substituted phthalocyanine zinc are then collected.

2) Fine separation:
a) Balancing through a fine separation column: the fine separation column has a packed length of 800 mm and a diameter of 150 mm. The fillers are Japanese Daisogel C₁₈ 10 μm;
b) Injection: take the roughly separated substances to make a 62% DMF aqueous solution with pH 8.0. The aqueous solution is pumped into the fine separation column. In the above solution, the content of dualistic substituted phthalocyanine zinc is 0.10% of filler weight. The concentration of dualistic substituted phthalocyanine zinc is 7 g/L. The injection column temperature is 28° C. The injection flow rate is 150 ml/min;
c) Elution: pH 8.0˜8.2, 62% DMF aqueous solution is used as a flowing phase for elution in the fine separation column. The eluting column temperature is 28° C. The elution flow rate is 450 ml/min. According to the formula of the eluent used here, four isomer-enriched fractions of dualistic substituted phthalocyanine zinc are collected;
d) Solvent Replacement: The fractions obtained at the step 2 c) are pumped into a replacement column. The fillers are the same as those used in for fine separation column. A 70% acetonitrile aqueous solution, having the same pH value as that used for injection, is used for elution. Enriched fractions of dualistic substituted phthalocyanine zinc are then collected.

### Example 3

The synthetic products are unchanged.

1) Rough separation:
a) Balancing through a rough separation column: the rough separation column has a packed length of 1000 mm and a diameter of 200 mm, and fillers are Capcell PaR C₁₈ G-80 50 μm;
b) Injection: take the synthetic products to make 65% DMF aqueous solution of pH 7.0 to 8.0, and pump the DMF solution into the rough separation column. In the above solution, the content of contains dualistic substituted phthalocyanine zinc content is 0.35% of the filler weight. The concentration of dualistic substituted phthalocyanine zinc is 15 g/L. Injection column temperature is 35° C. The injection flow rate=800 ml/min.
c) Elution: The eluting column temperature is 28° C. The remaining conditions are the same as those in Example 1.
d) Column enrichment: The conditions are the same as those in Example 1, except that a 20% DMF aqueous solution and a 75% (volume) DMF aqueous solution are used in turns for elution. The enriched fractions of dualistic substituted phthalocyanine zinc are then collected.

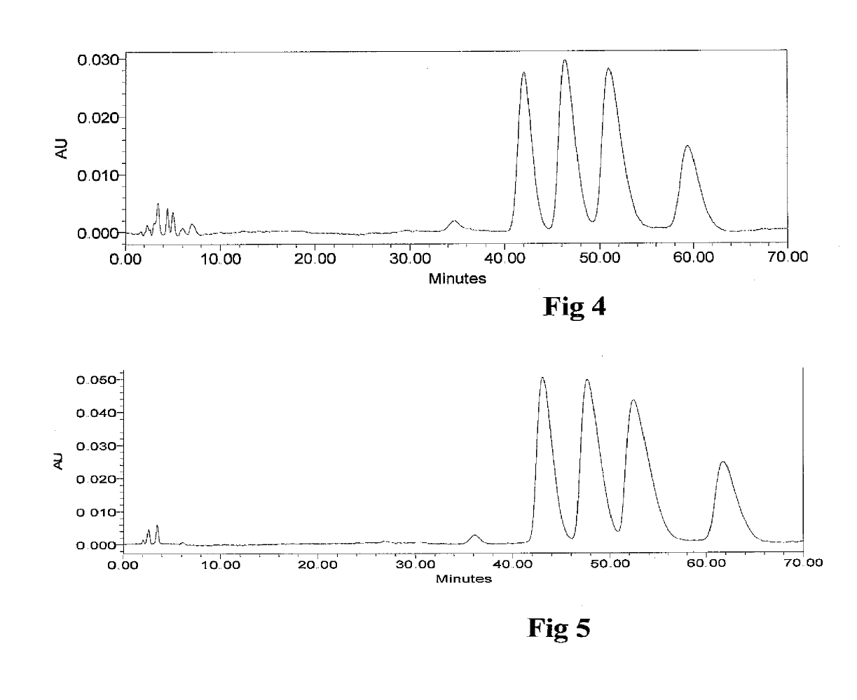

2) Fine Separation:
a) Balancing through a fine separation column: the fine separation column has a packed length of 1000 mm and a diameter of 200 mm. The fillers are Japanese Capcell PaR C₁₈ UG-80 20 μm;
b) Injection: take the roughly separated substances to make a 65% DMF aqueous solution with pH 8.0. The aqueous solution is pumped into the fine separation column. In the above solution, the content of dualistic substituted phthalocyanine zinc is 0.12% of filler weight. The concentration of dualistic substituted phthalocyanine zinc is 10 g/L. The injection column temperature is 35° C. The injection flow rate is 400 ml/min;
c) Elution: pH 8.0˜8.2, 65% DMF aqueous solution is used as a flowing phase for elution in the fine separation column. The eluting column temperature is 35° C. The elution flow rate is 400 ml/min. According to the formula of the eluent used here, four isomer-enriched fractions of dualistic substituted phthalocyanine zinc are collected. The HPLC graphs of the collected fractions are shown in FIG. 5;
d) Solvent Replacement: The fractions obtained at the step 3 c) are pumped into a replacement column. The fillers for the replacement column are the same as those used in for fine separation column. A 5% acetonitrile aqueous solution and a 75% acetonitrile aqueous solution, both having the same pH value as that used for injection, are used for elution in turns. Thereafter, 5% acetonitrile aqueous solution is used first for 5 cycles before the use of the 75% acetonitrile aqueous solution. The target enriched fractions are then collected.

The present invention has been described via the detailed illustration of the preferred embodiment. Those skilled in the art can derive variations from the preferred embodiment without departing from the scope of the present invention. Therefore, the preferred embodiment shall not limit the scope of the present invention defined in the claims.

What is claimed is:

1. A dualistic substituted phthalocyanine zinc formula, characterized in that the above dualistic substituted phthalocyanine zinc formula includes the following four isomers of phthalocyanine zinc: 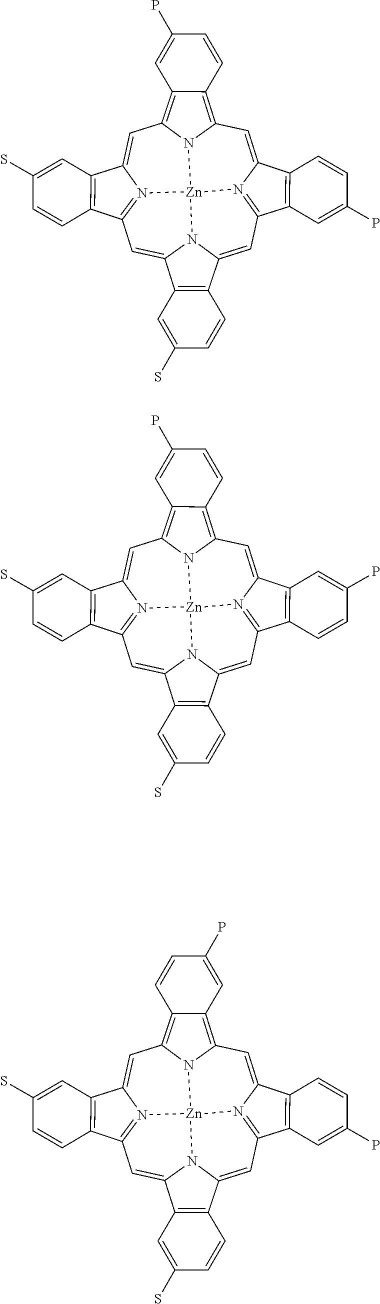 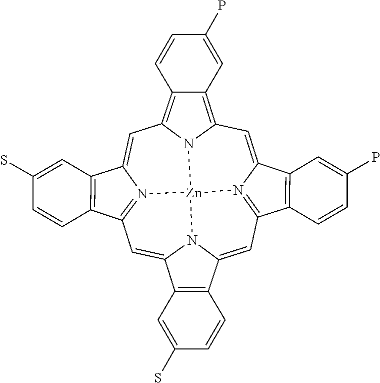
the above dualistic substituted phthalocyanine zinc formula are isomers of ZnPcS₂P₂, wherein Zn is divalent zinc ions, Pc is phthalocyanine ring base, —S is —SO₃⁻M⁺, and SO₃⁻ is sulfo-group, M⁺ is a pharmaceutically acceptable alkali metal cation,
—P is

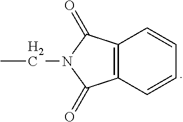

2. An use of a dualistic substituted phthalocyanine zinc formula used in photodynamic therapy for cancer or precancerous lesions, characterized in that the above dualistic substituted phthalocyanine zinc formula includes the following four isomers of phthalocyanine zinc:
the above dualistic substituted phthalocyanine zinc formula are isomers of ZnPcS₂P₂, wherein Zn is divalent zinc ions, Pc is phthalocyanine ring base, —S is —SO₃⁻M⁺, and SO₃⁻ is sulfo-group, M⁺ is a pharmaceutically acceptable alkali metal cation,
—P is

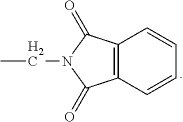

3. The use of claim 2, characterized in that the above M⁺ is potassium ion.

4. A separation process of dualistic substituted phthalocyanine zinc formula, characterized in that the process includes the following steps:
Step 1: separating ten cis isomers of ZnPcS₂P₂ from synthetic products comprised of ZnPcS₄, ZnPcS₃P, ZnPcS₂P₃, and ZnPcP₄, wherein Zn is divalent zinc ions, Pc is phthalocyanine ring base, S is —SO₃⁻M⁺, and SO₃⁻ is sulfo-group, M⁺ is a pharmaceutically acceptable alkali metal cation,
P is 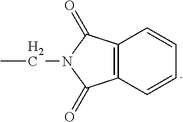
Step 2: separating the above four isomers of dualistic substituted phthalocyanine zinc of claim 1 from the above ten cis isomers of ZnPcS₂P₂.

5. A separation process of dualistic substituted phthalocyanine zinc formula of claim 4, characterized in that the separation process use industrial chromatographic column separation method, wherein chromatographic column fillers are revers-phase C₁₈ silica gels, the above fillers pore diameter is 100 A to 120 A, the above fillers carbon load is 17% to 19%, the above fillers diameter is 10-50 um, column temperature is 20° C. to 35° C., the eluant is a 65%˜70% (volume) DMF aqueous solution with buffer, the above eluant pH is 7.0 to 8.0.
